(19) United States
(12) Patent Application Publication
(10) Pub. No.: US 2009/0105720 A1
(43) Pub. Date: Apr. 23, 2009

(54) Non-invasive surgical tenaculum

(76) Inventor: Brenda J. Boone, Virginia Beach, VA (US)

Correspondence Address: GUERRY LEONARD GRUNE, 784 S VILLIER CT., VIRGINIA BEACH, VA, 23452, US

(21) Appl. No.: 11/975,425

(22) Filed: Oct. 19, 2007

(51) Int. Cl. A61B 17/42

(52) U.S. Cl. 606/119

(57) ABSTRACT

Disclosed is a tanaculum for surgery and examination of bio-tissue wherein the tenaculum has a first lever having a first disk on the distal end and the second lever having a second disk on the distal end, the first disk and the second disk having a textured surface on each face in opposition to each other wherein there is no sharp surface in contact with bio-tissue to prevent perforation, impaling, tearing or penetrating the tissue.

### FIELD OF THE DISCLOSURE

The present invention relates to medical devices, and more particularly, to a manually operated tenaculum useful in the manipulation and examination of a female cervix.

### BACKGROUND OF DISCLOSURE

Certain surgical and diagnostic devices are known for examining vaginal and other cavities. For example, women of a certain age should have a diagnostic pap smear annually. Certain prior art devices, known as speculums which consist of a pair of wide or broad blades are used in conjunction with other instruments which are uncomfortable to the patient, causing much discomfort.

Additionally, when such instruments are used in examining a body cavity, such as the vagina, other instruments are inserted into the vagina through the speculum. One such instrument is called a lateral vaginal retractor and is used to retract the vaginal side walls which normally obstruct the doctor's view into the patient's cervix. During a surgical procedure, the surgeon may insert a clamp, a source of lighting, a suctioning catheter, and irrigation tube through the speculum, thus further obstructing the surgeon's view of the cervix which might interfere with the surgical procedure being performed.

The tenaculum, usually has two pivotally mounted arms having inwardly and opposing directed spikes for hysteroscopic examination. The spikes are placed so as to grip the cervix allowing for positioning of the cervix. Gripping action is performed by the spikes impaling the tissue of the cervix, and the lack of impedance of such impaling causes bleeding, trauma and pain for the patient. It is sometimes necessary to use two of these tenacula, which creates awkwardness, hinders mobility and puts the patient at greater risk of bleeding and infection.

The use of manipulative instruments in the course of examination of a patient, or in the course of a surgical procedure, are well known in the art. Thus, various forceps and retention devices have been developed in the past which, in one way or another, conform to the physiology of a person.

Specific amongst these prior art devices are various forceps or tenacula of more or less general application and also various retention tools and forceps conformed for particular aspects of the patient's anatomy.

What is needed is a tenaculum for cervical examination that eliminates piercing, tearing and injury to the cervical tissue.

### RELEVANT ART

U.S. Pat. No. 5,059,198, to Gimpelson, Richard J., and unassigned describes a gynecological tenaculum for gripping the exterior of the cervix of the human uterus to press the cervix inwardly against a sheath for a hysteroscope inserted through the cervix canal to resist refluxing of distension media within the uterus through the cervix canal around the sheath. At includes a pair of movable extension members pivotally associated with each other; each extension member comprising a trunk and a pair of arms branching distally from each trunk, the length of each arm being greater than the length of the trunk from which it branches. The arms grip the exterior of the cervix to press the cervix inwardly against the sheath to resist the refluxing of distension media through the cervix canal and around the sheath due to each arm having an enlarged pressure distribution section near its anterior end. Each distribution section has a surface facing inwardly from the distribution section positioned for pressing inwardly against the cervix, and a tooth projecting inwardly from each distribution surface for engaging the cervix and pressing the cervix inwardly against the sheath. Each pair of arms has an arm that is substantially straight until it reaches a point near the distribution section with the substantially straight arms being in alignment with one another.

U.S. Pat. No. 5,868,668, to Weiss, Sol, and unassigned describes a surgical instrument for carrying out diagnostic and therapeutic procedures on the interior organs of the human body having a handle with a pair of integral spaced flanges at the upper end. The spaced flanges have a pair of spaced apertures and a first blade fixed to the flanges extending outwardly. A flange housing mounted to the spaced flanges also has a second blade fixed extending outwardly and spaced from and above the first blade. There are a pair of levers, with each lever having a pivot pin receivable in one of the apertures through the flanges pivotally mounting the levers to the flanges and each of the levers having a blade integral and extending outwardly. Each blade mounted to the levers is disposed one above the other between the first and second blades and adapted to move from a first position lying in a plane generally parallel to the planes of the first and second blades. When the first and second blades are disposed adjacent to each other and moved to a second position, lying generally a plane vertically disposed with respect to the planes of the first and second blades, the latter are moved apart and the flange housing has an opening for receiving a surgical instrument.

U.S. Pat. No. 5,499,997, to Sharpe, et. al., and assigned to Sharpe Endosurgical Corp.describes an endoscopic grasping tool surgical instrument for grasping tissue. There is a control handle, having a foregrip and a rear grip; a tubular sheath coupled to the control handle; a connector rod coupled to the control handle, adapted for reciprocating motion within the tubular sheath, a first jaw arm, having a first tooth for engaging tissue and a second jaw arm, having a second tooth. The first jaw arm and second jaw arm are coupled to the connector rod and adapted to be moved to an extended position by motion of the connector rod. They are also adapted to be moved to a retracted position by motion of the connector rod. The first jaw arm has a distal first contact pad, the second jaw arm has a distal second contact pad moving toward the retracted position placing the distal first contact pad and the distal second contact pad into contact. The first tooth and the second tooth intermesh along a line of contact forming a blunt distal end. A spring located in the control handle provides biasing force to the first jaw arm and to the second jaw arm into retracted position. The first jaw arm and the second jaw arm have a substantially arcuate shape defining a first apex surface on the first jaw arm and defining a second apex surface of the second jaw arm. The first apex surface and the second apex surface are biased and in abutment to each other during motion toward the retracted position and a gas seal within the control handle marked concentrically abut the connector rod.

U.S. Pat. No. 5,329,943, to Johnson, Gerald W., and assigned to Jeffrey W. Johnson.describes a surgical abdominoplasty procedure eschewing the use of large abdominal incisions and sutures by making a small incision through the umbilicus, inserting a surgical cutting and cauterizing instrument through the incision and undermining the skin over a sufficient area of the fascia and muscle to be repaired to permit smooth contraction of the skin over the repaired fascia and muscle. The surgical clamping instrument is inserted through the incision, underneath the skin and above the fascia, to engage the fascia at predetermined spaced points. The clamping instrument operate to plicate the fascia by inserting a fascial stapler through the incision and stapling the plicated fascia with surgical staples and repeating the procedure at a predetermined spacing along the fascia to complete the fascial and muscular repair and suturing or closing the umbilical incision.

U.S. Pat. No. 5,655,544, to Johnson, Gerald W., and unassigned describes a surgical abdominoplasty procedure eschewing the use of large abdominal incisions making at least one small hidden incision under the armpit, in a pre-existing scar, on the abdomen or in the umbilicus of inside the pubic hair line. A surgical cutting and cauterizing instrument is inserted through the incision and undermining the skin over a sufficient area of the fascia and muscle to be repaired to permit smooth contraction of the skin over the repaired fascia and muscle. The surgical clamping instrument inserted through the incision, underneath the skin and above the fascia engages the fascia at predetermined spaced points. Operating the clamping instrument plicates the fascia and inserting a suturing needle and suture through the incision and suturing the plicated fascia, repeating the procedure at a predetermined spacing completes the fascial and muscular repair by and suturing or closing the small incision.

U.S. Pat. No. 5,336,228, to Cholhan, Hilary J., and unassigned describes a tenaculum useful in the manipulation and excision of a cervix and uterus having a first forcep piece characterized by a first handle, a first generally cylindrical arm extending from the first handle. The first arm extends into a pair of first split tines at the free end, diverging from the arm to form a tapered separation with each first tine formed and twisted to align the free edges along first parts of a circle. There is a second forcep piece characterized by a second handle, a second generally cylindrical arm extending from the second handle and aligned for receipt between the first tines within the interior of the tapered separation proximate the juncture with the first arm. The pair of second tines at the free end of the second arm include, a pair of second tines formed as a split structure bent and twisted to align the free edges along a second part of the circle. A pivot extends through the first and the second arm for pivotal engagement including a first and second ball seat formed in the first tines, a first and second ball receivable in the corresponding ones of the ball seat and a pivot rod connecting the first and second ball. Pivotal motion of the second arm into the juncture between the first tines affects the flexure reducing the spacing between the free ends of the first tines.

U.S. Pat. No. 5,582,577, to Lund, et. al., and assigned to Vance Products Inc.describes a surgical retractor, retainer, tensioner, tenaculum, spreader or stabilizer particularly adapted for facile introduction into and removal from the body through a laparoscopic surgical cannula during a laparoscopic procedure. It has a plurality of tissue-engaging elements, an elastic member connecting the plurality of tissue-engaging elements that is collapsible into and through the laparoscopic surgical cannula. The plurality of tissue-engaging elements are shaped and dimensioned so as to allow the retractor to be passed through the laparoscopic surgical cannula into and out of the body, so that the retractor can be contained completely within the body during the laparoscopic procedure.

U.S. Pat. No. 5,645,561, to Smith, et. al., and assigned to Utah Medical Products, Inc.describes a hand-held gynecologic instrument with a rigid elongate body having a proximate end and a distal end. A manipulation member pivotally connects to the elongate body at the distal end, a handle connected to the proximate end of the elongate body with at least a portion of the handle angularly disposed from the elongate body acting as an actuating lever pivotally connected to the instrument adjacent the handle. The actuating lever is disposed and configured to allow the same hand to simultaneously hold the handle and pivot the actuating lever. A rod, having a first end pivotally connected to the actuating lever and having a second end pivotally connected to the manipulation member such that the rod translates pivotal movement of the actuating lever with respect to the elongate body into pivotal movement of the manipulation member with respect to the elongate body. The manipulation member has an appendage having a tip end and a securement end and a pivot block with an opening for receiving the securement end of the appendage. The pivot block is pivotally connected to the rod and also pivotally connected to the elongate body at the distal end. There is an appendage securing, in releasable engagement, the securement end of the appendage within the opening of the pivot block. The tip end of the appendage is a curved portion and the appendage securement releasably secures the appendage within the opening of the pivot block at a plurality of rotational orientations and at various distances from the elongate body.

U.S. Pat. No. 7,048,744, to Weiss, Sol, and unassigned describes an attachment device for use with surgical clamps that rests on the handle of such clamps while maintaining the position and/or tension of such clamps having a generally unshaped elongated body adapted to overlie a portion of the handle of the clamp. Additionally, a plurality of stabilizing hooks are affixed to and extending from the elongated body to assist in maintaining the engagement of the elongated body onto the clamp. The elongated body is formed in two cooperating engageable sections and lockable whereby the overall length of the elongated body can be selectively chosen and maintained.

U.S. Patent Publication No. 2002/0165579A1, to Burbank, et. al., and unassigned describes a device useful for compressing a uterine artery of a patient having a handle with a proximal end and a distal end and a compressing portion mounted to the handle distal end with the compressing portion having a distal end face and a side surface.

U.S. Patent Publication No. 2002/0177842A1, to Weiss, Sol, and unassigned describes an attachment device for use with surgical clamps that rests on the handle of such clamps while maintaining the position and/or tension of such clamps with a generally unshaped elongated body adapted to overlie a portion of the handle of the clamp and a plurality of stabilizing hooks affixed to and extending from the body to assist in maintaining the engagement of the body onto the clamp.

U.S. Patent Publication No. 2005/0113634A1, to Burbank, et. al., and assigned to Vascular Control Systems, Inc.describes an intravaginal device for occluding a female patient's uterine arteries with an unsymmetrical anatomy to treat a uterine disorder. There is a first occluding member which has a first elongated shaft having a first operative proximal shaft section configured to extend out of the patient during treatment. There is a first distal shaft section with a first pressure applying occluding element secured to the first distal shaft section having a first mechanism to adjust the orientation or the location of at least part of the occluding element with respect to the first distal shaft section. Also a second occluding member with a second elongated shaft having a second operative proximal shaft section configured to extend out of the patient during treatment and which has a second distal shaft section with a second pressure applying occluding element secured to the second distal shaft section. The connection between the first and second occluding members is configured to adjust spacing between the first and second occluding elements to press the pressure applying elements against the patient's vaginal wall to occlude underlying uterine arteries.

U.S. Patent Publication No. 2004/0158262A1, to Burbank, et. al., and assigned to Vascular Control Systems, Inc.describes an intravaginal device for delivery of a medical instrument to a female patient's uterine cervix, related tissue or near-by anatomical structure. An elongated guide rail has a distal portion with a distal tip, which has a proximal portion with a free proximal end configured to receive and guide a medical instrument to the patient's uterine cervix, related tissue or near-by anatomical structure. Also, a tissue grasping mechanism secured to the distal portion of the guide rail configured to grasp the patient's uterine cervix to facilitate delivery of the medical instrument over the guide rail to the patient's uterine cervix.

U.S. Design Patent No. 321055, to Carchidi, Joseph E., and assigned to Ace Surgical Supply, Inc.describes an ornamental design for a caliper for dental implants.

WIPO Publication No. 02065924, to Vanheerden, Marcus Vincent, and unassigned describes a uterine manipulator device for injecting a visualization fluid into the uterus of a female via the cervix, the uterine manipulator device has an elongate body that can be inserted into the uterus of a female via the cervix having a visualization fluid conduit along which the visualization fluid can be conveyed to the uterus and an outlet opening for the visualization fluid. Connected to the elongate body is a means for releasably gripping the cervix and a sealing means that acts between the elongate body and the cervix so as to form a fluid-resistant seal between the elongate body and the cervix thereby preventing a return-flow of visualization fluid.

WIPO Publication No. 02078522, to Burbank, et. al., and assigned to Vascular Control Systems, Inc.describes a device useful for compressing a uterine artery of a patient having a handle with a proximal end and a distal end and a compressing portion mounted to the handle distal end with the compressing portion having a distal end face and a side surface.

### SUMMARY OF THE DISCLOSURE

Disclosed is a tanaculum for surgery and examination of a cervix wherein the tenaculum has a first lever and a second lever, singlarily hinged pivotably together near a mid-point along their length with a first lever having a first loop on a proximal end and the second lever having a second loop on the proximal end for grasping and the first lever having a first disk on the distal end and the second lever having a second disk on the distal end, the first disk and the second disk having a textured surface on each face in opposition to each other wherein there is no sharp surface in contact with human or bio-tissue.

An additional embodiment is a tenaculum wherein the disk surface is from 0.125″ to 0.500″ in diameter and may be grooved, cross-hatched, etched, coated or sand blasted to produce a texture.

An additional embodiment is a tenaculum wherein the textures surface of the first and second disk allows for clamping of the cervix without perforation, impaling, tearing or penetrating the tissue.

Another embodiment is a tenaculum having a holding feature wherein clamping of the cervix at a desired pressure may be maintained.

Another embodiment is a tenaculum having a first lever and a second lever that may be straight, curved or angled along their length.

Another embodiment is a tenaculum having loops on the proximal end to aid in grasping.

### BRIEF DESCRIPTION OF THE DRAWINGS

FIG. 1A is a tenaculum for intravaginal examination.

FIG. 1B is a detail of the textured surface of the disk.

### DETAILED DESCRIPTION OF THE DRAWINGS

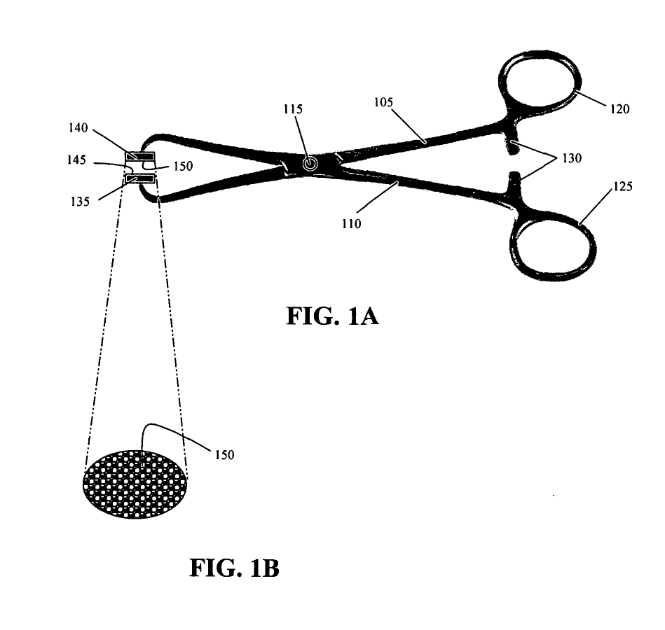

FIG. 1A shows a tenaculum [100] with a first lever [105] and a second lever [110] with a hinge [115] (shown as a pin) connecting the first lever [105] and second lever [110]. The first lever [105] and/or the second lever [110] may be bent or curved for a particular application. On the proximal end of the first lever [105] is a first loop [120] and on the proximal end of the second lever [110] is a second loop [125] for grasping. Additionally, near the proximal end of the first lever [105] and the second lever [110] is an interjoining holding feature [130] which allows for maintaining a desired clamping pressure of the human tissue to be examined.

On the distal end of the first lever [105] is a first disk [135] and on the distal end of the second lever [110] is a second disk [140] which may be round or ellipsoid in shape creating a first disk face surface [145] and a second disk face surface [150] having no sharp contours that form opposing surfaces. The first disk face surface [145] and a second disk face surface [150] having a textured surface (Detailed in FIG. 1B) that allows for gripping of a cervix without perforation, impaling, tearing or penetrating the tissue.

In operation, the medical personnel (not shown) would place a finger in a first loop [120] and a thumb in the second loop [125] in order to manipulate the tenaculum [100]. The first disk surface [145] and the second disk surface [150] are placed on either side of the cervix to be examined or operated upon. The first lever [105] and second lever [110] are then squeezed together to create a pressure on the tissue with the first disk surface [145] and the second disk surface [150] gripping the tissue sufficiently. The holding feature [130] may then be employed to maintain gripping contact with the tissue.

FIG. 1B illustrates a typical surface texture of the second disk face surface [150]. The first disk surface [145] would have a similar texture. The texture shown in FIG. 1B is for illustration purposes.

1. A tanaculum for surgery and examination of a cervix comprising; a first lever and a second lever, singlarily hinged pivotably together near a mid-point along their length with said first lever having a first loop on a proximal end and said second lever having a second loop on a proximal end; said first lever having a first disk on a distal end and said second lever having a second disk on a distal end, said first disk and said second disk having a textured surface on each face in opposition to each other wherein there is no sharp surface in contact with human tissue.

2. The tanaculum for surgery and examination of a cervix of claim 1, wherein said first disk and said second surface is from 0.125″ to 0.500″ in diameter and may be grooved, cross-hatched, etched or sand blasted to produce a texture.

3. The tanaculum for surgery and examination of a cervix of claim 1, wherein said textures surface of said first disk and said second disk allows for clamping of human tissue without perforation, impaling, tearing or penetrating the tissue.

4. The tanaculum for surgery and examination of a cervix of claim 1, wherein said tenaculum has a holding feature wherein clamping of said cervix at a desired pressure may be maintained.

5. The tanaculum for surgery and examination of a cervix of claim 1, wherein said first lever and said second lever may be straight, curved or angled individually along their length.

6. The tanaculum for surgery and examination of a cervix of claim 1, wherein said tenaculum has loops on the proximal end to aid in grasping.

7. A tanaculum for surgery and examination of bio-tissue comprising; a first lever and a second lever, singlarily hinged pivotably together near a mid-point along their length with said first lever having a first loop on a proximal end and said second lever having a second loop on a proximal end; said first lever having a first disk on a distal end and said second lever having a second disk on a distal end, said first disk and said second disk having a textured surface on each face in opposition to each other wherein there is no sharp surface in contact with human tissue.
